(19) United States
(12) Patent Application Publication
(10) Pub. No.: US 2017/0056463 A1
(43) Pub. Date: Mar. 2, 2017

(54) COMPOSITIONS AND METHODS FOR TREATING DIABETES

(71) Applicant: Suzy Cohen, Broomfield, CO (US)

(72) Inventor: Suzy Cohen, Broomfield, CO (US)

(21) Appl. No.: 15/254,364

(22) Filed: Sep. 1, 2016

Related U.S. Application Data
(60) Provisional application No. 62/212,920, filed on Sep. 1, 2015.

(51) Int. Cl. A61K 36/53; A61K 31/355; A61K 31/455; A61K 31/519; A61K 31/194; A61K 31/4741; A61K 31/05; A61K 36/185; A61K 9/00; A61K 9/48; A61K 9/20; A23L 33/00; A23L 33/16; A23L 33/15; A23L 33/105; A61K 31/593

(52) U.S. Cl. CPC A61K 36/53; A61K 31/593; A61K 31/355; A61K 31/455; A61K 31/519; A61K 31/194; A61K 31/4741; A61K 31/05; A61K 36/185; A61K 9/0053; A61K 9/48; A61K 9/20; A23L 33/40; A23L 33/16; A23L 33/15; A23L 33/105; A23V 2002/00

(57) ABSTRACT

The present disclosure is a compound, a method of making the compound and method of using such compound preferably in the form of a dietary supplement that, when administered, is capable of treating prediabetes, diabetes, insulin resistance or metabolic syndrome. The unique combination of the composition is preferably administered orally. The composition is preferably comprised of at least vitamin D, gamma tocopherol, niacin, folate, chromium, zinc, magnesium, potassium, bilberry, resveratrol, holy basil and berberine, in pre-determined amounts. The composition can further comprise a palliative agent, and can be provided in the form of a capsule, powder, liquid or tablet.

### CROSS-REFERENCE TO RELATED APPLICATION

This application claims priority to and the benefit under 35 U.S.C. §119(e) to U.S. Provisional Patent Application Ser. No. 62/212,920 filed Sep. 1, 2015, which is incorporated by reference in its entirety.

### FIELD OF THE INVENTION

The present invention is directed to compounds, particularly dietary supplements, and methods for formulating and administering the same.

### BACKGROUND OF THE INVENTION

The human pancreas is both a gland and an organ, and makes up an important part of the human digestive tract. The pancreas is best known for its secretion of insulin, a hormone that reduces blood glucose (sugar). Insulin is a pancreatic hormone whose primary task is to assist in blood sugar regulation in the human body. Insulin resistance occurs when your body cannot use circling insulin properly, allowing your blood sugar levels to rise. Insulin resistance is a precursor to type 2 diabetes, as well as a risk factor in many other chronic diseases. The pancreas is part of the human endocrine system. The pancreas also secretes hormones, including glucagon, somatostatin (a.k.a. growth hormone) as well as pancreatic polypeptide. These secretions all circulate within the human bloodstream.

The pancreas is also a digestive organ, and secretes a pancreatic juice that helps you digest food. This “juice” contains strong digestive enzymes to assist in the break down and absorption of nutrients in the small intestine. These enzymes further help to break down carbohydrates, proteins, and lipids from the meals you eat.

Type 2 diabetes is formerly known as non-insulin-dependent diabetes mellitus (NIDDM). Type 2 diabetes is also known as “Adult Onset Diabetes.” Insulin is made in a section of the pancreas where a cluster of cells called “Beta Cells” (or (β-cells) reside. Pancreatic dysfunction (poor (β-cell function) is the primary cause of Type 2 diabetes, due to reduced insulin secretion, absorption or utilization by the human body.

The pancreas regulates the amount of insulin produced, and also produces pancreatic hormones (mentioned above) which break down fats, proteins and carbohydrates from the foods when eaten. In turn, the speed at which the food metabolizes these nutrients affects how these compounds produce energy. The amount of insulin produced and secreted by (β-cells in the pancreas impact the amount of blood sugar shuttled into the cells.

In addition to these functions, pancreatic hormones have several critical functions in the human body, especially pertaining to human metabolic and cardiovascular systems. For example, production of insulin promotes the storage of fat in adipocytes, increases DNA replication, and forces storage of glucose in liver cells and muscle cells. When insulin resistance (or other pancreatic problems) develop in the human body, blood glucose rises, and left unchecked and untreated, this situation causes systemic inflammation due to elevated blood glucose. These and related problems have resulted in an epidemic, with the estimated number of people with diagnosed and yet-to-be diagnosed diabetes at approximately 29.1 million people in the United States alone, which equates to approximately 9.3 percent of the population.

There are presently a number of known but unresolved problems relating to diabetes and similar pancreas-related disorders. By way of example, elevated blood glucose can cause a person to feel disabling nerve pain, to have cardiac palpitations, to have uncontrolled appetite, and to retain weight. Other health and associated problems are known to those of ordinary skill in the art. Complications of untreated diabetes, and particularly metabolic syndrome, often cause complications such as kidney failure, blindness, heart attacks, obesity, hypertension, hypercholesterolemia and amputations.

These problems and others are addressed by the compositions and methods described in detail below.

### SUMMARY OF THE INVENTION

The invention relates to a compound, particularly in the form of a dietary supplement, which improves upon the healthy function of the pancreas, and insulin-producing β-cells.

In varying embodiments described herein, the invention relates to a compound that is capable of treating pre-diabetes, diabetes, metabolic syndrome or other pancreas-related disorders. Certain elements of the novel compounds and methods for formulating the same are described in varying levels of detail herein.

In addition to the problems associated with blood sugar abnormalities described herein, obese people are also more susceptible to nutrient deficiencies. This deficiency can lead to blood sugar dysregulation and elevated insulin hormone. Through experimentation it has been found that including various minerals, such as chromium, zinc, magnesium and potassium can be beneficial to people who have conditions that adversely affect their metabolism (hypertension, hypercholesterolemia, hypertriglyceridemia, metabolic syndrome, obesity, pre-diabetes, insulin resistance, etc.).

More particularly, people who are deficient in chromium have unstable blood sugars. Chromium can improve insulin metabolism. For example, Chromium has been demonstrated to inhibit phosphotyrosine phosphatase, the enzyme that would otherwise cleave phosphate from the insulin receptor, and eventually lead to decreases in insulin sensitivity and thus, higher blood glucose. Chromium can also enhance insulin binding to the cell, insulin receptor number, insulin internalization, and β-cell sensitivity. Through experimentation, the Applicant has found that compounds containing chromium, in particular a highly absorbable “chelated” form of chromium (by way of example but not limitation, chromium nicotinate glycerinate chelate) can help a person with pre-diabetes, diabetes or other disorders with treating insulin and blood glucose abnormalities. As for obesity, common with diabetes, chromium supplementation increases muscle gain and fat loss, particularly when combined with exercise, and improves glucose metabolism and as well as the serum lipid (cholesterol) profile in patients with or without diabetes.

Dietary supplements containing berberine are able to reestablish, improve and reduce blood glucose levels in patients with pre-diabetes and diabetes. After oral supplementation with the herbal extract of berberine, the levels of fasting blood sugar have been found to normalize.

Therefore, it would be desirable to include berberine in a dietary supplement in order to positively influence human metabolism of blood sugar, and more specifically reduce elevated levels of blood glucose.

A chromium deficiency can further contribute to a situation where the human pancreatic gland has an insufficient ability to produce insulin, and can cause a person to develop elevated blood glucose, pre-diabetes, diabetes, metabolic syndrome or other undesirable effects. Therefore, there is a need for a dietary supplement that promotes the health benefits of minerals, including chromium (as well as magnesium, potassium, and zinc as described in greater detail herein) and assists certain people to retain these minerals, particularly in combination with berberine, in order to control blood sugar and reduce complications of diabetes.

Resveratrol is beneficial to treating diabetes and insulin resistance. Resveratrol supplementation reduces fasting blood sugar, hemoglobin A1c (another blood sugar parameter) and also reduced painful sensations of diabetic neuropathy, a complication of diabetes. Resveratrol can also significantly reduce inflammatory cytokines that can target the pancreas and ultimately reduce insulin production. Therefore, providing a composition that also comprises appropriate levels of resveratrol can also be beneficial.

Other nutrients and compounds can provide further benefits to both the pancreas and adrenal glands of the human body, and are described in greater detail herein.

Methods for formulating the compound are also disclosed herein. The unique combination of the composition is preferably administered orally in the form of a capsule. The unique combination has synergistic advantages over previously known compositions. As disclosed in more detail in the Detailed Description, the present invention provides compositions, methods for treating pre-diabetes, diabetes, metabolic syndrome or other pancreas-related disorders, and methods for forming the compound.

The composition is preferably comprised of a unique and novel formulation in pre-determined amounts, and further provides benefits previously unexpected. In addition to other health benefits described herein, the composition:
enhances the production of insulin;
provides for peripheral utilization of insulin;
provides for reduction of blood glucose and hemoglobin A1c; and
assists the human body in optimizing metabolism.

Further, this formula is provided by way of a non-glandular formula, which comprises supportive nutrients for a healthy pancreas gland.

In certain embodiments, the composition provides additional nutrients necessary for reduction of blood sugar and pro-inflammatory cytokines (compounds), which target the human pancreas.

In a preferred embodiment, the composition is comprised of at least vitamin D3, gamma tocopherol, niacin, folate, magnesium, zinc, chromium, potassium and a blend of herbal components, by way of example including but not limited to, berberine, Holy Basil Leaf, resveratrol, bilberry or combinations/sub-combinations thereof. These ingredients can be included, which promote healthy metabolism and pancreatic function as well as healthy adrenal gland function within the human body, and as described in detail herein.

More particularly, a preferred embodiment of the present disclosure comprises the following, with variability in dosages listed below:

1) Vitamin D3—between about 1,000 IU and about 10,000 IU;

2) Vitamin E—between about 20 and about 200 mg;

3) Niacin—between about 5 mg and about 500 mg;

4) Folate—between about 25 mcg and about 1,000 mcg;

5) Magnesium—between about 50 mg and about 500 mg;

6) Zinc—between about 1 mg and about 40 mg;

7) Chromium—between about 100 mcg and about 500 mcg; and

8) Potassium—between about 30 mg and about 300 mg.

In some embodiments, the blend can contain any one of the following ingredients, alone or in any combination:

9) Berberine—between about 50 mg and about 1,000 mg;

10) Holy Basil Leaf—between about 10 mg and about 1,000 mg;

11) Trans-resveratrol—between about 25 mg and about 800 mg; and

12) Bilberry—between about 10 mg and about 400 mg.

In one embodiment, the composition is provided as a dietary supplement. In one embodiment, the composition is administered in the form of a capsule. In an alternate embodiment, the composition is administered in the form of a gummy chew, tablet, powder or liquid extract. In further embodiments, the composition comprises one or more palatability agents to favorably alter the taste of the composition for human consumption.

An aspect of the invention is a composition for treating diabetes-related disorders. The composition comprises between about 1,000 and about 10,000 IU of vitamin D3, between about 20 to about 200 mg of vitamin E, between about 5 and about 500 mg of niacin, between about 25 mcg and about 1,000 mcg of folate, between about 50 and about 500 mg of magnesium, between about 1 and about 40 mg of zinc, between about 100 and about 500 mcg of chromium, between about 30 and about 300 mg of potassium, between about 50 and about 1,000 mg of berberine, between about 10 and about 1,000 mg of Holy basil leaf, between about 25 and about 800 mg of resveratrol, and between about 10 and about 400 mg of bilberry.

An aspect of the invention is a composition for treating diabetes, the composition comprises about 1,000mg of vitamin D, about 300 IU of gamma tocopherol, about 20 mg of niacin, about 200 mg of folate, about 125 mg of magnesium malate, about 5 mg of zinc bisglycinate chelate, about 250 mcg of chromium nicotinate glycerinate chelate, about 90 mg of tripotassium citrate monohydrate, about 25 mg of bilberry, about 150 mg of berberine, about 50 mg of Holy Basil extract, and about 50 mg of a trans isomer of resveratrol.

An aspect of the invention is a method to treat diabetes. Diabetes is treated in the patient by administering a composition comprising between about 1,000 and about 10,000 IU of vitamin D3, between about 100 and about 800 IU of vitamin E, between about 5 and about 500 mg of niacin, between about 25 mcg and about 1,000 mcg of folate, between about 50 and about 500 mg of magnesium, between about 1 and about 40 mg of zinc, between about 100 and about 500 mcg of chromium, between about 30 and about 300 mg of potassium, between about 50 and about 1,000 mg of berberine, between about 10 and about 1,000 mg of Holy basil leaf, between about 25 and about 800 mg of resveratrol, and between about 10 and about 400 mg of bilberry.

These and other advantages will be apparent from the disclosure of the invention(s) contained herein. The above-described embodiments, objectives, and configurations are neither complete nor exhaustive. The Summary of the Invention is neither intended nor should it be construed as being representative of the full extent and scope of the present invention.

References made herein to “the present invention” or aspects thereof should be understood to mean certain embodiments of the present invention and should not necessarily be construed as limiting all embodiments to a particular description. The present invention is set forth in various levels of detail in the Summary of the Invention and the Detailed Description, and no limitation as to the scope of the present invention is intended by either the inclusion or non-inclusion of elements in this Summary of the Invention. Additional aspects of the present invention will become more readily apparent from the Detailed Description and appended claims.

### DETAILED DESCRIPTION

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

As used herein, the phrases “at least one”, “one or more”, and “and/or”, as used herein, are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Unless otherwise indicated, all numbers expressing quantities, dimensions, conditions, and so forth used in the specification and claims are to be understood as being modified in all instances by the term “about”.

The term “a” or “an” entity, as used herein, refers to one or more of that entity. As such, the terms “a” (or “an”), “one or more” and “at least one” can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

It shall be understood that the term “means” as used herein shall be given its broadest possible interpretation in accordance with 35 U.S.C. Section 112(f). Accordingly, a claim incorporating the term “means” shall cover all structures, materials, or acts set forth herein, and all of the equivalents thereof. Further, the structures, materials, or acts and the equivalents thereof shall include all those described in the summary of the invention, brief description of the drawings, detailed description, abstract, and claims themselves.

### Composition:

An aspect of the present invention is a composition for treatment of diabetes. The components of the composition listed below can be used in any sub-combination. Thus, the sub-combinations of components are embodiments of the invention.

### Vitamin D

In a preferred embodiment, the composition is comprised of vitamin D. In a most preferred embodiment, the compound is comprised of a pre-determined amount of the activated form of vitamin D known as “D3” or “cholecalciferol.” It has been found from experimentation that this form of vitamin D is optimal for human absorption. As autoimmune antibodies are known to attack and damage the endocrine glands (including the pancreas) of certain individuals, it is desirable to include a pre-determined amount of D3 with the composition, which can reduce autoimmune attack. Vitamin D helps improve insulin sensitivity and responsiveness by the cell (thus improving blood glucose levels). In addition, certain individuals have a deficiency in this fat-soluble vitamin D, and therefore have trouble regulating blood glucose and controlling their immune response. A deficiency of vitamin D can cause DNA damage, blunt the immune response, exasperate cardiovascular problems, and cause increased permeability within the intestinal tract, thus intensifying the immune response, and raising blood glucose which in turn attacks the human pancreas gland.

In a preferred embodiment, the composition comprises from about 1,000 to 10,000 mg of Vitamin D, preferably cholecalciferol. In a most preferred embodiment, the composition comprises about 1,000 mg of cholecalciferol. In alternate embodiments, one or more additional fat-soluble vitamins are included or substituted for cholecalciferol.

### Vitamin E

Accumulating evidence suggests that reactive oxygen species (ROS) known more commonly as “oxidative stress” is part of the cytokine cascade that leads to dangerous complications in diabetes. Antioxidant agents, particularly, fat-soluble ones like natural Vitamin E are thought to ameliorate some of the free radical damage. Gamma tocopherol, an isomer of natural Vitamin E, benefits people with type 2 diabetes. It is a gentle platelet inhibitor meaning it can thin blood. People with diabetes often have microvascular complications and thinning blood can reduce cardiovascular complications of diabetes and metabolic syndrome. Gamma tocopherol can reverse or delay the onset of insulin resistance and resultant pathogenesis.

In alternate embodiments, one or more additional fat-soluble vitamins are included or substituted for vitamin E. In some embodiments, fat-soluble vitamins can be used in combination with vitamin E. Fat-soluble vitamins include, but are not limited to, vitamin A and vitamin K. In some embodiments, various forms of vitamin E can be combined and used with this invention. By way of example only, a blend of vitamin E is EVNol™. This blend, or other blends including vitamin E, can be used with this invention.

In a preferred embodiment, the composition comprises from about 20 to about 200 mg of vitamin E, preferably gamma tocopherol. In a most preferred embodiment, the composition comprises about 95 mg of gamma tocopherol.

### Niacin

Niacin is also known as Vitamin B3. B vitamins are important for the heart, and cardiac complications are rather common in patients with diabetes and metabolic syndrome. Niacin is helpful to reduce homocysteine, which when elevated can increase risk of heart attack. Niacin amide, a niacin amide, can be used as it does not cause skin flushing, or elevate uric acid levels. Niacin is used to help support the body's natural detoxification pathway, the methylation pathway. A healthy methylation pathway will help produce important compounds, which in turn help improve insulin production, and reduce blood glucose.

In a preferred embodiment, the composition comprises from about 5 to 500 mg of niacin. In a most preferred embodiment, the composition comprises about 20 mg of niacin amide.

### Folate

Folate is another B vitamin known as vitamin B9. Folate is a natural body-ready, highly bioavailable form of vitamin B9 which is needed for the human body's natural detoxification pathway known as the “methylation” pathway. In some embodiments, folic acid can be used in place of folate. Folic acid is similar to folate, but is not methylated, making it more difficult for the body to break down. Thus, in some embodiments, folic acid cannot be used in place of folate.

Folate goes by many names. The form of folate used in a preferred composition is referred to as “Metafolin™” and is a high-quality methylated form of folate known chemically as 5 methyl tetrahydrofolate (5MTHF). Metafolin™ is different from common folic acid, which may be found in other dietary supplements.

Folate is known to support nerve health, thus reducing sensations of diabetic neuropathy in people with diabetes. Folate is also included to improve methylation, a detoxification pathway that is often impaired in patients with diabetes and insulin resistance. The reason for this impairment varies, and is sometimes caused by genetic polymorphisms in the MTHFR pathway. Other times it is impaired due to nutritional deficiencies due to use of medications (either pharmaceuticals or over-the-counter). Including folate (with niacin) will improve methylation in the human body, and the utilization of insulin.

Folate is also included to reduce homocysteine, a pro-inflammatory compound in the human body that is sometimes elevated in people with diabetes. When elevated, homocysteine can raise a person's risk for heart attack, a common complication of untreated diabetes.

In a preferred embodiment, the composition comprises from about 25 to 1000 mcg of folate. In a most preferred embodiment, the composition comprises about 200 mcg of folate (aka Metafolin™)

In alternate embodiments, one or more additional natural B vitamins are included or substituted for folate. Suitable natural B vitamins including B6 (also known as pyridoxine) can be used with the invention. The amount of B6 can be between about 10 to 100 mg. A more biologically active form of B6 called P5P (pyridoxal 5 phosphate), which is a metabolite of pyridoxine, in the range of about 1 to about 25 mg can also be used with the invention.

Vitamin B12 in the range of 50 mcg to 3,000 can also be used with the composition. There are many forms of B12. Forms of B12 include, but are not limited to, methylcobalamin, adenosylcobalamin, hydroxocobalamin or cyanocobalamin.

### Magnesium

Magnesium is an important ion and critical to all living cells, especially in glucose homeostasis. Magnesium can be in the form of magnesium malate.

Magnesium is a key electrolyte which can be affected by blood glucose disorders. When electrolytes are out of normal range, cardiac arrhythmias can result. Investigation has shown that higher magnesium intake reduces risk of impaired glucose and insulin metabolism, and slows progression from the pre-diabetic state to full blown diabetes in middle-aged subjects. Magnesium intake can be particularly beneficial in offsetting risk of developing diabetes. Higher magnesium intake can improve insulin sensitivity and reduce type 2 diabetes risk.

Low intracellular levels of magnesium can result from increased urinary excretion of magnesium, or from insulin resistance. Reduced magnesium can result in impaired insulin response and higher blood glucose levels. Magnesium deficiencies caused by diabetes can also upset the body's potassium levels, hence potassium is also in this formula. Poorly controlled diabetes can cause magnesium deficiencies, which can in turn cause hypokalemia, or low potassium levels. Individuals with hyperglycemia resulting from poorly controlled diabetes can benefit from taking magnesium supplements (which can also help correct potassium imbalances).

Magnesium can also be used in a supplement form to improve cardiovascular health, which is often compromised in people with prediabetes, diabetes and metabolic syndrome. Magnesium intake can be a significant protective factor against type 2 diabetes, especially among those with insulin resistance, low-grade inflammation or a drinking habit.

In a preferred embodiment, the composition comprises from about 50 to 500 mg of magnesium. In a most preferred embodiment, the composition comprises about 125 mg of magnesium.

### Zinc

Zinc is required to convert proinsulin to insulin, forming a zinc-insulin hexamer intracellularly. Thus, zinc is a mineral that improves levels of insulin in the human body. Zinc deficiencies impair the utilization of insulin. Blood glucose levels were reduced in 2 hour post-prandial measurements in people treated with zinc. Further investigation shows a significant reduction in systolic and diastolic blood pressures after zinc supplementation, thus reducing risk to the diabetic of cardiovascular complications and hypertension (common in diabetes).

Zinc is an antioxidant mineral that supports immune function. Zinc deficiency can play a role in people with abnormal immune function in Type 2 diabetes according to research. In fact, people with diabetes are known to have insufficient amounts of zinc in their blood, and higher amounts in their urine. Independent investigation suggests that appropriate levels of zinc in the blood are linked to slower risk of diabetes. Zinc is also known to protect the skin. Skin infections which progress to gangrene and can required amputation, a rather common complication of diabetes and metabolic syndrome. Zinc is important to speed wound healing and critical to those with abnormal blood sugar and pancreatic gland problems.

In a preferred embodiment, the composition comprises from about 1 to 40 mg of zinc. In a most preferred embodiment, the composition comprises about 5 mg of zinc bisglycinate chelate.

### Chromium

This mineral is often used to help people maintain blood sugar levels and control appetite. People with Type 2 diabetes who take chromium supplements can exhibit improved hemoglobin A1c values, fasting blood glucose levels and blood sugar control. Chromium is sometimes used by body builders because it is thought to improve lean muscle and reduce body fat. Thus, it can play a role in those with obesity. Other details regarding the influence of chromium on subjects with pre-diabetes, diabetes or metabolic syndrome are provided in the Summary of the Invention above.

Previous investigation has been performed to assess chromium handling in non-insulin dependent diabetic patients (NIDDM). Significant differences in chromium homeostasis were seen between patients and controls. Plasma chromium values were inversely correlated with plasma glucose, suggesting that chromium has a positive effect on fasting blood glucose.

In a preferred embodiment, the composition comprises from about 100 to 500 mcg of chromium, preferably chromium nicotinate glycerinate chelate. In a most preferred embodiment, the composition comprises about 250 mcg of chromium. In alternate embodiments, one or more additional minerals are included or substituted for chromium.

### Potassium

Potassium remains in homeostasis with other minerals in the human body, including magnesium. Both magnesium and potassium are needed for healthy blood pressure. Controlling blood pressure is important for people with diabetes, as hypertension increases risk for cardiovascular complications. In addition, potassium is an electrolyte which can be affected by blood glucose disorders. Potassium can be in the form of tripotassium citrate monohydrate.

In a preferred embodiment, the composition comprises from about 30 to 300 mg of potassium. In a most preferred embodiment, the composition comprises about 90 mg of tripotassium citrate monohydrate.

Bilberry (Vaccinium myrtillus)

Bilberry can also be known by other names, including huckleberry, or European blueberry. The natural blue pigment is a strong antioxidant that quells free radicals.

Bilberry is best known to impact eyesight. Blindness can be a complication of diabetes. Independent investigation has shown that bilberry extract lowered blood glucose and increased insulin sensitivity by activating AMP-activated protein kinase (AMPK) in white adipose (fat) tissue, skeletal muscle and the liver. AMPK stimulates fat breakdown in the tissue, liver and muscle, and modulates insulin secretion by your pancreas.

In alternate embodiments, one or more additional herbal extracts can be included or substituted for bilberry. Suitable herbal extracts include, but are not limited to, black currant extract (amount in the composition between about 100 and about 500 mg), Vinpocetine 5 (amount in the composition of about 10 mg), beta carotene (amount in the composition of about 5,000 to 20,000 IU), vitamin A (amount in the composition of about 3 to 10,000 IU), grape seed (amount in the composition of about 25-100 mg), blueberry extract (amount in the composition of about 100-500 mg), or ginkgo biloba (amount in the composition of about 100 to 250 mg).

In a preferred embodiment, the composition comprises from about 10 to 400 mg of bilberry. In a most preferred embodiment, the composition comprises about 25 mg of bilberry.

### Berberine

In a preferred embodiment, the composition is comprised of a pre-determined amount of “berberine,” a known botanical among practitioners of Chinese and Ayurvedic medicine. This compound is found in the Oregon grape leaf, and has been found through experimentation to reduce blood glucose. Berberine can lower blood glucose in doses of about 1,000 mg daily as effectively as metformin, a popular prescription medicine for diabetes. In previous investigations, berberine caused a significant reduction in hemoglobin A1c (HbA1c) and improved cholesterol ratios. Additional investigations suggest that berberine can prevent pre-diabetes from developing. Berberine has antiviral activity, and inhibits growth of HSV (herpes simplex virus). It has been shown to reduce inflammation chemicals in the brain.

In a preferred embodiment, the composition comprises from about 50 to 1000 mg of berberine. In a most preferred embodiment, the composition comprises about 150 mg of berberine.

### Holy Basil

Holy Basil is also known as “Tulsi” and has an adaptogenic effect on adrenal glands and the ability to impact cortisol rhythm. Because of its remarkable effect on cortisol secretion, glucose regulation and lipids, Holy Basil is included in this composition. A reduction of cortisol leads to a higher sense of well-being and relaxation. Holy Basil has been found through experimentation to support normal levels of cortisol, which then supports healthy blood pressure and lipid metabolism.

Holy Basil has strong anti-inflammatory properties. It has a stabilizing effect on blood sugar and cholesterol ratios. Animal models have shown benefits to post-prandial blood glucose levels and fasting blood glucose. Holy Basil is protective to the liver, which is where the human body stores glycogen (stored blood sugar). Additionally, this herb has demonstrated antifungal, antibacterial and antiviral properties, important for the human endocrine system.

In a preferred embodiment, the composition comprises from about 10 to 1000 mg of Holy Basil, standardized to contain 2% ursolic acid (an active compound). In a most preferred embodiment, the composition comprises about 50 mg of Holy Basil extract.

### Resveratrol

Resveratrol is a powerful antioxidant found naturally in red grapes, berries and cacao. Resveratrol has been found to be beneficial to treating diabetes and insulin resistance. Recent experimentation has shown that resveratrol supplementation reduced fasting blood sugar, hemoglobin A1c (another blood sugar parameter) and also reduced painful sensations of diabetic neuropathy, which can be a complication of diabetes. Resveratrol can also significantly reduce inflammatory cytokines that can target the pancreas and ultimately reduce insulin production. Therefore, providing a composition that also comprises appropriate levels of resveratrol would be beneficial.

Resveratrol improves insulin sensitivity. It improves thyroid function, which in turn supports blood sugar regulation. It protects eyesight and other visual disturbances, which can lead to blindness, which can be a complication of high blood glucose. Resveratrol is also thought to help a person attain healthy cholesterol ratios, thus reducing the risk of cardiovascular complications often associated with diabetes. In the case of general diabetic complaints, resveratrol can help relieve pain, fatigue and inflammation. This antioxidant is a powerful free-radical scavenger, which can also help reduce risk for cancer.

In a preferred embodiment, the composition comprises from about 25 to 800 mg of resveratrol. A trans isomer or a cis isomer of resveratrol can be used. In a most preferred embodiment, the composition comprises about 50 mg of resveratrol, using the trans isomer.

### Absent Elements

In addition, the composition of a preferred embodiment is substantially free of the following common allergens: milk, casein, eggs, shellfish, tree nuts, peanuts, wheat, gluten, and soybeans. The composition preferably does not contain artificial colors, flavors, or preservatives, and is free from magnesium stearate (a common lubricant used in the manufacture of pharmaceuticals and dietary supplements).

### Additional Elements

In addition to the benefits described herein, several of the botanicals and nutrients described in varying embodiments of the present invention have been found to exhibit antiviral, antifungal and antibacterial activity against a wide variety of organisms, which in turn provides an additional layer of protection to the human pancreatic gland and endocrine system.

In varying embodiments, the composition can further comprise variances in regards to encapsulation, or powder formulations. According to certain embodiments, the compositions described herein can further be provided with one or more palatability agents. These palatability agents serve to add flavor to the composition so that an effective dosage is easier to be ingested. It is within the scope of the present invention that any safe, flavor enhancing palatability agent can be used in a composition of the present invention. Particularly suitable palatability agents for use in the composition of the present invention include, but are not limited to, plant oils, plant hydrolysates, yeast, yeast hydrolysates, and combinations thereof.

### Methods

An aspect of the invention is a method to treat diabetes with a composition comprising vitamin D3, vitamin E, niacin, folate, magnesium, zinc, chromium, potassium, berberine, Holy basil leaf, resveratrol, and bilberry. The patient is treated by providing an effective amount of the composition. By way of example, between 1 and 3 capsules of the composition can be provided to a patient per day, in some embodiments 2 capsules per day. The capsules can be taken by the patient daily, preferably in the morning before any other food is ingested by the patient.

An aspect of the invention is a method to prepare a compound to treat diabetes. The method comprises providing proportional amounts of each material such that the resulting composition results in between about 1,000 and about 10,000 IU of vitamin D3, between about 100 and about 800 IU of vitamin E, between about 5 and about 500 mg of niacin, between about 25 mcg and about 1,000 mcg of folate, between about 50 and about 500 mg of magnesium, between about 1 and about 40 mg of zinc, between about 100 and about 500 mcg of chromium, between about 30 and about 300 mg of potassium. In some embodiments, the mixture can further comprise between about 50 and about 1,000 mg of berberine; between about 10 and about 1,000 mg of Holy basil leaf; between about 25 and about 800 mg of resveratrol; and between about 10 and about 400 mg of bilberry. The components are mixed, then can be provided to a delivery device (for example capsule).

While various embodiments of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims. For further illustration, the book Diabetes Without Drugs (Rodale 2010) authored by the named inventor of the present invention is expressly made a part of this disclosure and incorporated by reference herein in their entirety.

Ranges have been discussed and used within the forgoing description. One skilled in the art would understand that any sub-range within the stated range would be suitable, as would any number within the broad range, without deviating from the invention.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

What is claimed is:

1. A composition for treating diabetes-related disorders, comprising:
between about 1,000 and about 10,000 IU of vitamin D3;
between about 20 to about 200 mg of vitamin E;
between about 5 and about 500 mg of niacin;
between about 25 mcg and about 1,000 mcg of folate;
between about 50 and about 500 mg of magnesium;
between about 1 and about 40 mg of zinc;
between about 100 and about 500 mcg of chromium;
between about 30 and about 300 mg of potassium;
between about 50 and about 1,000 mg of berberine;
between about 10 and about 1,000 mg of Holy basil leaf;
between about 25 and about 800 mg of resveratrol; and
between about 10 and about 400 mg of bilberry.

2. The composition of claim 1, wherein the composition is provided as a dietary supplement.

3. The composition of claim 1, wherein the composition is administered orally in the form of a capsule.

4. The composition of claim 1, wherein the composition is administered orally in the form of a tablet.

5. The composition of claim 1, wherein the composition is substantially free of at least one of milk, casein, egg, shellfish, tree nut, peanut, wheat, gluten, artificial flavors, artificial colors, preservative, magnesium stearate, and soybean.

6. The composition of claim 1, further comprising at least one palatability agent, and wherein the palatability agent is selected from the group consisting of a plant oil, a plant hydrolysate, yeast, a yeast hydrolysate, and combinations thereof

7. The composition of claim 1, wherein the vitamin D is cholecalciferol, and comprising about 1,000 mg of cholecalciferol.

8. The composition of claim 1, wherein the vitamin E is gamma tocopherol, and the composition comprises about 300 IU of gamma tocopherol.

9. The composition of claim 1, comprising about 20 mg of niacin.

10. The composition of claim 1, wherein the folate is Metafolin™, and wherein the composition comprises about 200 mg of the Metafolin™

11. The composition of claim 1, wherein the magnesium is magnesium malate, and wherein the composition comprises about 125 mg of magnesium malate.

12. The composition of claim 1, wherein the zinc is zinc bisglycinate chelate, and wherein the composition comprises about 5 mg of zinc bisglycinate chelate.

13. The composition of claim 1, wherein the chromium is chromium nicotinate glycerinate chelate, and wherein the composition comprises about 250 mcg of chromium nicotinate glycerinate chelate.

14. The composition of claim 1, wherein the potassium is tripotassium citrate monohydrate, and wherein the composition comprises about 90 mg of tripotassium citrate monohydrate.

15. The composition of claim 1, comprising about 25 mg of bilberry.

16. The composition of claim 1, comprising about 150 mg of berberine.

17. The composition of claim 1, comprising about 50 mg of Holy Basil extract.

18. The composition of claim 1, wherein the resveratrol is a trans isomer, and the composition comprises about 50 mg of the trans isomer of the resveratrol.

19. A composition for treating diabetes, comprising:
about 1,000 mg of vitamin D;
about 300 IU of gamma tocopherol;
about 20 mg of niacin;
about 200 mg of folate;
about 125 mg of magnesium malate;
about 5 mg of zinc bisglycinate chelate;
about 250 mcg of chromium nicotinate glycerinate chelate;
about 90 mg of tripotassium citrate monohydrate;
about 25 mg of bilberry;
about 150 mg of berberine;
about 50 mg of Holy Basil extract; and
about 50 mg of a trans isomer of resveratrol.

20. A method to treat diabetes, comprising:
administering the following to a patient:
between about 1,000 and about 10,000 IU of vitamin D3;
between about 20 to about 200 mg of vitamin E;
between about 5 and about 500 mg of niacin;
between about 25 mcg and about 1,000 mcg of folate;
between about 50 and about 500 mg of magnesium;
between about 1 and about 40 mg of zinc;
between about 100 and about 500 mcg of chromium;
between about 30 and about 300 mg of potassium;
between about 50 and about 1,000 mg of berberine;
between about 10 and about 1,000 mg of Holy basil leaf;
between about 25 and about 800 mg of resveratrol; and
between about 10 and about 400 mg of bilberry.
